(19) United States
(12) Patent Application Publication
(10) Pub. No.: US 2013/0041292 A1
(43) Pub. Date: Feb. 14, 2013

(54) Customizable Haptic Assisted Robot Procedure System with Catalog of Specialized Diagnostic Tips

(71) Applicant: James S. Cunningham, Boulder, CO (US)

(72) Inventor: James S. Cunningham, Boulder, CO (US)

(73) Assignee: TYCO Healthcare Group LP, Boulder, CO (US)

(21) Appl. No.: 13/205,969

(22) Filed: Aug. 9, 2011

(51) Int. Cl. A61B 18/12; A61B 18/18; A61B 18/00

(52) U.S. Cl. 601/2; 606/34; 606/33

(57) ABSTRACT

In accordance with the present disclosure, a system and method for using a remote control to control an electrosurgical instrument, where the remote controlled (RC) electrosurgical instrument has a universal coupling mechanism to allow switching between an interchangeable catalog of diagnostic tools. A controller within the base of the RC electrosurgical instrument identifies the type of disposable tip attached to the base. The controller, then, activates necessary features for use with the identified tip and deactivates any unnecessary features. A surgeon uses a remote with at least one momementum sensor to control the RC electrosurgical instrument 10. The surgeon rotates his hand mimicking movements of a handheld electrosurgical instrument, the movements of which are translated and sent to the RC electrosurgical instrument. The surgeon may use an augmented reality (AR) vision system to assist the surgeon in viewing the surgical site.

### BACKGROUND

1. Technical Field

The present disclosure relates to a system and method for remotely controlling an electrosurgical instrument and, more particularly, to a remote control system that controls a robotic tool with an interchangeable catalog of diagnostic tools.

2. Background of Related Art

Minimally invasive surgical procedures typically employ small incisions in body cavities for access of various surgical instruments, including forceps, laparoscopes, scalpels, scissors, and the like. It is often the case that several surgical hands, such as several laparoscopic instrument and camera holders, are necessary to hold these instruments for the operating surgeon during the particular surgical procedure. With the introduction of robotic-assisted minimally invasive surgery (MIS) in recent years, hospitals worldwide have made significant investments in acquiring this latest technology for their respective facilities.

Thus, it is known to use robotic-assisted MIS when carrying out surgical operations. When surgery of this kind is performed, access to a subcutaneous surgical site is provided via a number (typically 3 to 5) of small (typically 5-12 mm) incisions, through which a surgical arm is manually passed. The surgical arms are then coupled to the surgical robotic instrument, which is capable of manipulating the surgical arms for performing the surgical operations, such as suturing or thermally cutting through tissue and cauterizing blood vessels that have been severed. The surgical arms thus extend through the incisions during the surgery, one of which incisions is used for supplying a gas, in particular carbon dioxide, for inflating the subcutaneous area and thus create free space at that location for manipulating the surgical instruments.

Therefore, open surgeries often require a surgeon to make sizable incisions to a patient's body in order to have adequate visual and physical access to the site requiring treatment. The application of laparoscopy for performing procedures is commonplace. Laparoscopic surgeries are performed using small incisions in the abdominal wall and inserting a small endoscope into the abdominal cavity and transmitting the images captured by the endoscope onto a visual display. The surgeon may thus see the abdominal cavity without making a sizable incision in the patient's body, reducing invasiveness and providing patients with the benefits of reduced trauma, shortened recovery times, and improved cosmetic results. In addition to the endoscope, laparoscopic surgeries are performed using long, rigid tools inserted through incisions in the abdominal wall.

However, conventional techniques and tools for performing laparoscopic procedures may limit the dexterity and vision of the surgeon. Given the size of the incisions, the maneuverability of the tools is limited and additional incisions may be required if an auxiliary view of the surgical site is needed.

One example of a robotic assisted MIS system is the da Vinci® System that includes an ergonomically designed surgeon's console, a patient cart with four interactive robotic arms, a high performance vision system, and instruments. The da Vinci® console allows the surgeon to sit while viewing a highly magnified 3D image of the patient's interior sent from the high performance vision system. The surgeon uses master controls on the console that work like forceps to perform the surgery. The da Vinci® system corresponds to the surgeon's hand, wrist, and finger movements into precise movements of the instruments within the patient's interior.

However, the da Vinci® system only allows a single user to use the console and controllers at one time. Additionally, the 3D image shown in the da Vinci® system can only be viewed by the surgeon sitting at the console which prevents other surgeon's from assisting the surgeon in determining the best procedure to perform the surgery or from showing students how to perform the surgery. Additionally, the da Vinci® system is large and cumbersome and oversized relative to the electrosurgical instruments used in the procedure.

### SUMMARY

In accordance with the present disclosure, a system and method for using a remote control to control an electrosurgical instrument, where the remote controlled (RC) electrosurgical instrument has a universal coupling mechanism to allow switching between an interchangeable catalog of diagnostic tools. A controller within the base of the RC electrosurgical instrument identifies the type of disposable tip attached to the base. The controller, then, activates necessary features for use with the identified tip and deactivates any unnecessary features. A surgeon uses a remote with at least one momementum sensor to control the RC electrosurgical instrument 10. The surgeon rotates his hand mimicking movements of a handheld electrosurgical instrument, the movements are translated and sent to the RC electrosurgical instrument. The surgeon may use an augmented reality (AR) vision system to assist the surgeon in viewing the surgical site.

According to embodiment of the present disclosure, a remote controlled electrosurgical instrument assembly that includes a base configured with a transducer and a drive assembly. The remote controlled electrosurgical instrument assembly further includes an arm connected to the base and a tip removeably coupled to the arm. Additionally, the remote controlled electrosurgical instrument assembly includes a remote control configured to send a plurality of instructions to the base to control motions of the tip and for the base to supply an electrical signal to the tip.

According to another embodiment of the present disclosure, a remote controlled electrosurgical instrument assembly includes a base configured with a transducer and a drive assembly, and a first and second aim each connected to the base. The remote controlled electrosurgical instrument assembly further includes a first tip removably coupled to the first arm, and the first tip includes an ultrasonic end effector. Additionally, the remote controlled electrosurgical instrument assembly includes a second tip removably coupled to the second arm, and the second tip includes a RF end effector. A remote control configured to send a plurality of instructions to the base to control motions of the first tip and second tip. The base supplies an ultrasonic signal to the first tip when instructed by the remote control and supplies a RF signal to the second tip when instructed by the remote control.

According to another embodiment of the present disclosure, a method for performing an electrosurgical procedure includes the step of inserting a remote controlled electrosurgical instrument within a patient. The remote controlled electrosurgical instrument is configured with a base, an arm, and a removable tip coupled to the arm. The method also includes the step of moving a remote in a manner substantially similar to movement of a handheld electrosurgical instrument. The remote is configured with at least one momentum sensor. Further, the method includes the step of sending information from the momentum sensor to the base to move the remote controlled electrosurgical instrument within the patient based on movements of the remote. Additionally, the method includes the steps of removing the tip from the arm and coupling a second tip to the arm. The method also includes the steps of reading a sensor within the second tip to identify the type of tip and deactivating functions within the base not used by the second tip.

### BRIEF DESCRIPTION OF THE DRAWINGS

Various embodiments of the present disclosure are described herein with reference to the drawings wherein:

FIG. 1 is a schematic diagram of a remote controlled surgical system in accordance with an embodiment of the present disclosure;

FIGS. 2A-2C are perspective views of different remotes used in accordance with an embodiment of the present disclosure;

FIGS. 3A-3B are perspective views of a remote controlled electrosurgical instrument in accordance with an embodiment of the present disclosure;

FIGS. 4A-4E are perspective views of coupling mechanisms in accordance with an embodiment of the present disclosure;

FIGS. 5A-5D are perspective views of removable tips in accordance with an embodiment of the present disclosure;

FIG. 6 is a schematic diagram of an electrosurgical instrument control system in accordance with an embodiment of the present disclosure;

FIG. 7 is a schematic diagram of an augmented controller system in accordance with an embodiment of the present disclosure;

FIG. 8 is a schematic diagram of an augmented controller system in accordance with an embodiment of the present disclosure; and

FIG. 9 is a flow diagram of a process for setting up and controlling an electrosurgical instrument with removable tips in accordance with an embodiment of the present disclosure.

### DETAILED DESCRIPTION

Particular embodiments of the present disclosure are described hereinbelow with reference to the accompanying drawings. In the following description, well-known functions or constructions are not described in detail to avoid obscuring the present disclosure in unnecessary detail.

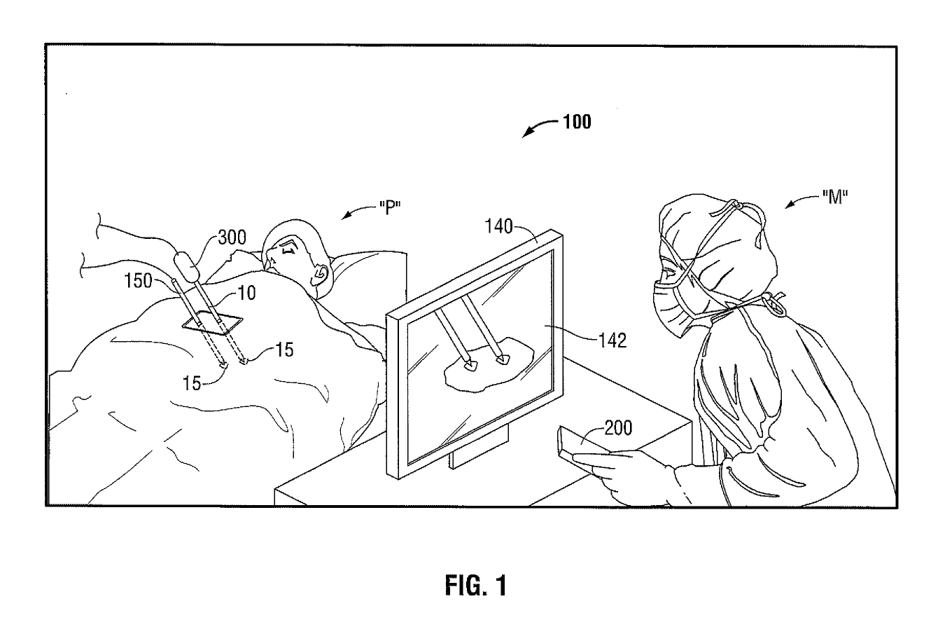

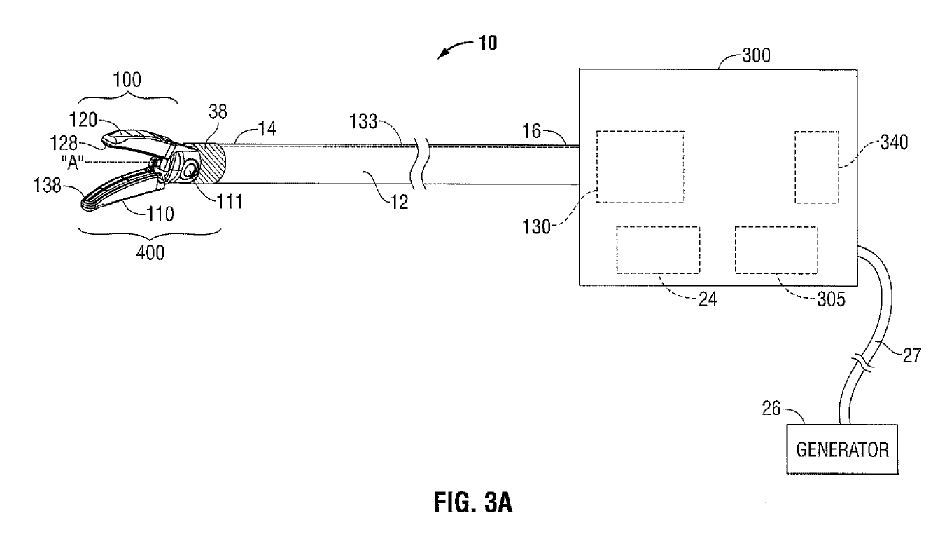

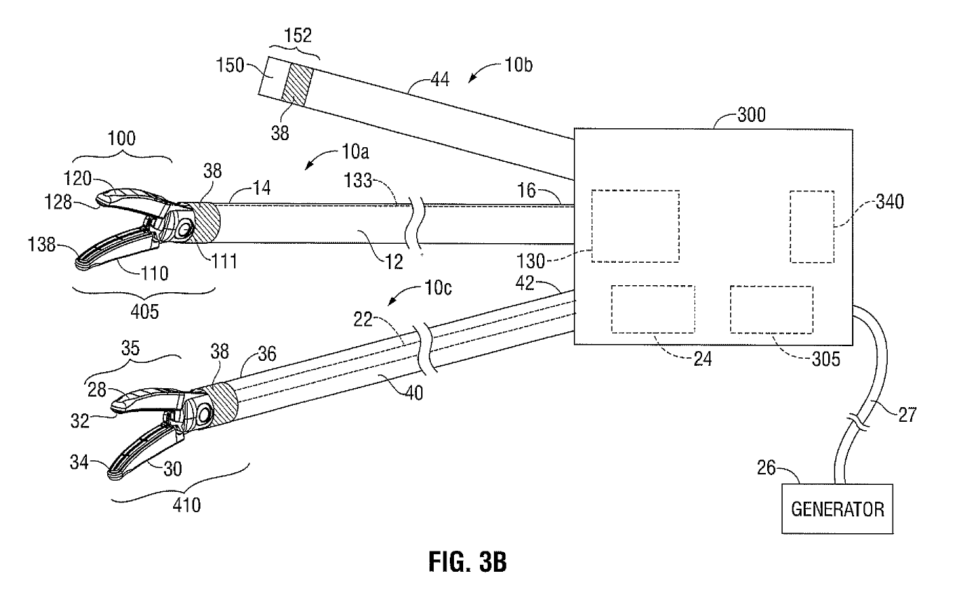

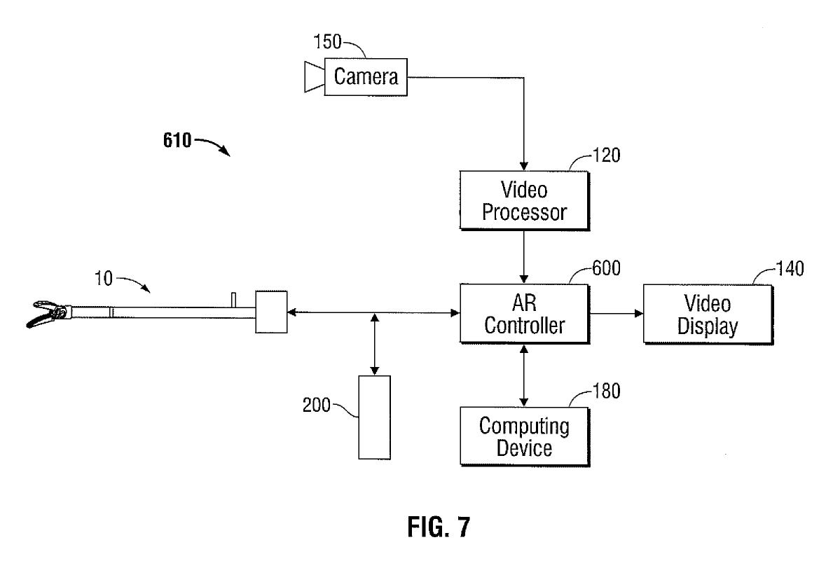

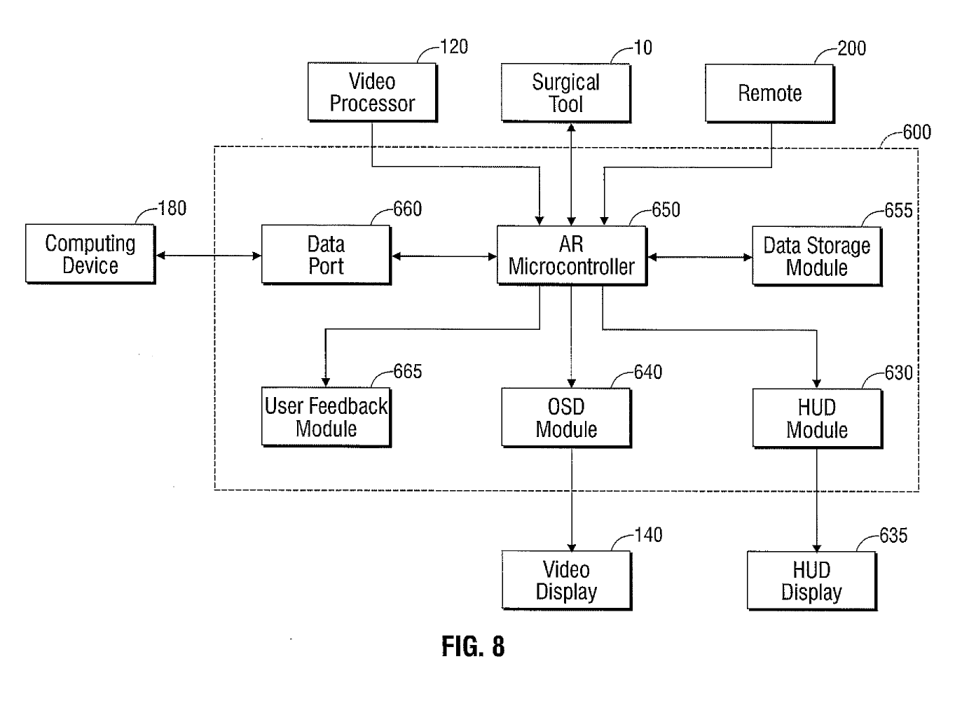

FIG. 1 is a schematic diagram of a remote controlled surgical system 100 that allows a surgeon M to perform a surgical procedure on patient P using a remote 200. Access to a subcutaneous surgical site within patient P is provided via a number (typically 3 to 5) of small (typically 5-12 mm) incisions 15, through which at least one remote controlled (RC) electrosurgical instrument 10 is manually passed. Additionally, a camera 150 is inserted in at least one incision 15 to give the surgeon M a view of the surgical site. Alternatively, camera 150 can be removable attachment coupled to base 300 (See FIG. 3B). The video signal from the camera may be sent to an Augmented Reality (AR) controller 600 (See FIGS. 7 and 8) to add additional data. The video signal and additional data are then displayed on a user interface 140. The AR displayed image 142 may include labels on instruments, labels and/or margins of organs, tumors, or other anatomical bodies, and/or boundary zones around delicate anatomical bodies. The AR displayed image 142 may be in 2D or 3D. As the camera 150 is moved around the surgical site, the labels and data overlaid onto the video image move to the appropriate location.

The surgeon M controls the RC electrosurgical instrument 10 by rotating and/or moving the remote 200 up, down, left, right, diagonally, and/or rotating. The movement of the remote 200 may be configured to move in a manner similar to a hand-held electrosurgical instrument. Additionally, the surgeon M can press a button on the remote 200 to activate an electrical signal to coagulate, cut tissue, staple tissue, or perform another function of the instrument. The surgeon M can be located in the same room as a patient or in a remote location such as another state or country. The remote 200 may be configured to send data to a base 300 attached to the RC electrosurgical instrument 10. The data may be sent to the base 300 through a direct electrical connection or by Bluetooth®, ANT3®, KNX®, ZWave®, X10® Wireless USB®, IrDA®, Nanonet®, Tiny OS®, ZigBee®, 802.11 IEEE, and other radio, infrared, UHF, VHF communications and the like.

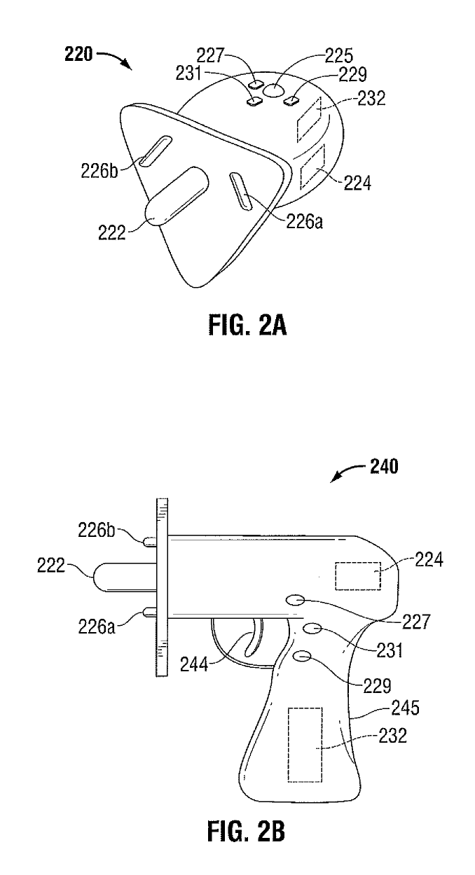

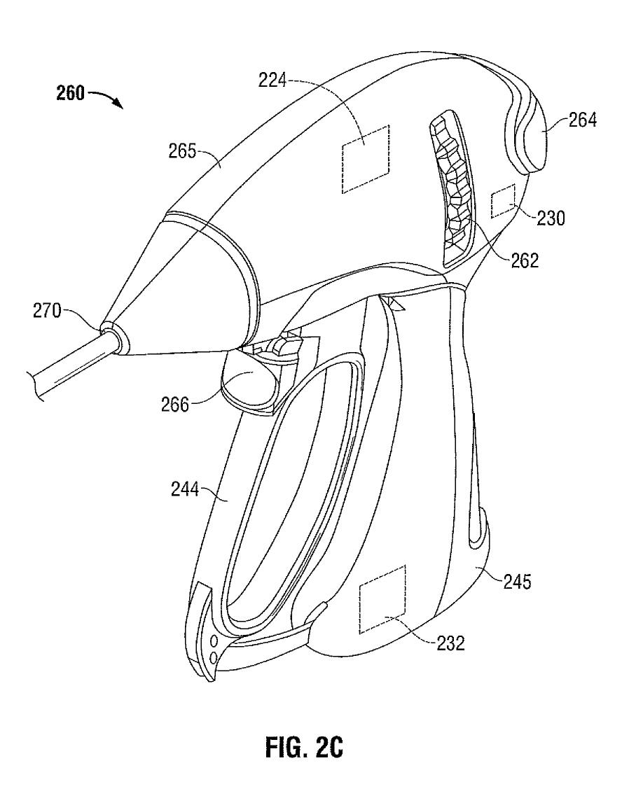

FIGS. 2A-C show three possible embodiments of remote 200, however, other embodiments may be possible. FIG. 2A discloses a first embodiment of a remote 220 that is generally circular in shape with a triangular front that may interconnect with the base 300 of the RC electrosurgical instrument 10. The circular shape allows the remote 220 to fit into the palm of the surgeon's M hand, where the surgeon M can rotate his/her wrist to move the tool in a corresponding manner by easily pushing one or more buttons 225, 227, 229, 231. The remote 220 includes at least one momentum sensor 224 and an infrared sensor 222. The remote may be configured with one or more buttons 225, 227, 229, 231 that may be located on the top, side, and/or bottom of the remote. Button 225 may be used to activate an electrical signal to coagulate, cut tissue, staple tissue, or perform other surgical functions. For example, button 227 may be used to move the end effector assembly 100 in very small increments. Additionally, the remote 220 includes a haptic feedback mechanism 232 that provides feedback about position, force used, instruction, and other similar uses. In an alternative embodiment, visual communication may be used to identify which instrument the remote is operating, problems with where the RC instrument 10 is located, battery life of remote, which remote in a master/slave relationship is controlling the instrument, and other problems with the RC instrument 10 or system. Alternatively, the remote 220 can be configured with audio feedback (not shown) to inform the surgeon M of problems or pre-recorded specific instrument functions. The remote 220 further includes data ports 226a and 226b for communicating with the instrument base 300. The data ports 226a and 226b may be connected directly to the instrument base 300 or wirelessly connected.

FIG. 2B discloses a second embodiment of a remote 240 for use with the remote controlled surgical system 100. Similar to the remote 220 in FIG. 2A, the remote 240 includes data ports 226a and 226b, momentum sensor 224, infrared sensor 222, and/or haptic feedback mechanism 232. Remote 240 is shaped with a handle 245 and a trigger 244. The trigger 244 is similar to button 225 on remote 220, and may be used to activate an electrical signal to coagulate, cut tissue, staple tissue, or perform another surgical function. Remote 240 further includes buttons 227, 229, and 231 used to perform other functions of the RC instrument 10. The size and shape of the handle 245 can be ergonomically shaped for a right-handed or left-handed surgeon and/or based on the size of the surgeon's hand.

FIG. 2C discloses a third embodiment of a remote 260. Similar to the remote 240 in FIG. 2B, the third remote 260 may include a housing 265, a momentum sensor 224, haptic feedback mechanism 232, handle 245, and/or trigger 244. Trigger 244 is similar to button 225 on remote 220, and may be used to activate an electrical signal to coagulate, cut tissue, staple tissue, or other procedure. Rotating wheel 262 is similar to button 227 on the first remote, and may be used to move the end effector assembly 100 in very small increments. Data port 230 wirelessly connects remote control 260 with the base 300 (see FIG. 3) of the RC electrosurgical instrument 10. Similar to the second remote 240, the size and shape of the handle 245 can be ergonomically shaped for a right-handed or left-handed surgeon and/or based on the size of the surgeon's hand. In alternative embodiments, remote 260 may also include opening 270 defined therein, where a surgeon can insert the same type end effector assembly 100 and shaft 12 as used within the patient P during surgery. This would allow the surgeon or others the ability see how the end effector is moving.

Referring to FIG. 3A, a RC surgical instrument 10, such as forceps, includes an arm 12 that has a proximal end 16 that mechanically engages the base 300 and a distal end 14 configured to mechanically engage a removable tip 400. The removable tip 400 includes an end effector assembly 100 and a coupling mechanism 38 to couple the removable tip 400 to arm 12. In the drawings and in the descriptions that follow, the term “proximal,” as is traditional, will refer to the end of the arm 12 which is closer to base 300, while the term “distal” will refer to the end that is farther from base 300. Alternatively, the system may be used with a remote controlled pencil or any other electrosurgical instrument.

Drive assembly 130 is in operative communication with the remote 200 through data port 340 for imparting movement of one or both of a pair of jaw members 110, 120 of end effector assembly 100. Drive assembly 130 may include a compression spring (not shown) or a drive wire 133 to facilitate closing the jaw members 110 and 120 around pivot pin 111. Drive wire 133 is configured such that proximal movement thereof causes one movable jaw member, e.g., jaw member 120, and operative components associated therewith, e.g., a seal plate 128, to move toward the other movable jaw member, e.g., jaw member 110 and seal plate 138. With this purpose in mind, drive rod or wire 133 may be made from any suitable material and is proportioned to translate within arm 12. In the illustrated embodiments, drive wire 133 extends through arm 12 past the distal end 14. Both jaw members 110 and 120 may also be configured to move in a bilateral fashion.

Base 300 receives an electrical signal from a generator 26. Generator 26 may be connected to base 300 by cable 27. By not including the generator 26 within base 300, the size of base 300 may be smaller. Additionally, base 300 may be used with an existing generator system. Alternatively, generator 26 may be part of base 300.

Remote control 200 (See FIG. 3A) may be in operative communication with an ultrasonic transducer 24 via data port 340 when the removable tip 400 is an ultrasonic tip 410 (See FIG. 3B). Alternatively, FIG. 3B shows base 300 with multiple arms 12, 44, 22. Each arm 12, 44, 22 may be removable or permanently attached to base 300. Also, as the surgical procedure is performed different arms 12, 14, 22 may be engaged or disengaged within patient P by the surgeon M using remote 200.

Each instrument 10a and 10c or camera attachment 10b coupled to base 300 may be controlled by a single remote control 200 or multiple remote controls. Instruments 10a and 10c each include an arm 12, 40 and a removable tip 405, 410, respectively. Instrument 10a is shown as RF bipolar tip 405 and instrument 10c is shown with an ultrasonic bipolar tip 440, but either may include any type of endoscopic tip. Any number of instruments 10a and 10c and or camera attachments 10b may be coupled to the base 300 at one time. The only limiting factor to the amount of attachments coupled to base 300 is the number coupling connectors (not shown) on base 300.

Instrument 10c includes removable tip 410 and arm 40 which is either removably coupled or permanently attached to base 300. Arm 40 is coupled at a proximal end 42 to base 300, and the distal end 36 of arm 40 is coupled to removable tip 410 via coupling mechanism 38. Arm 40 includes a length that ranges from about 20 cm to about 40 cm. In the illustrated embodiment, arm 40 includes a length that is 39 cm. Arm 40 may be rotated in a circular motion by hand movements of remote 200 or rotating a knob (not shown) on remote 200. A distal end 36 of arm 40 is operably coupled using coupling mechanism 38 to removable tip 410 and removable tip 410 includes end effector 35. The operation of parts of the end effector 35 (e.g., jaw members 28 and 30) are movable relative to one another upon actuation from remote control 200. More particularly, jaw member 28 is movable from an open position for positioning tissue between the jaw members 28 and 30, to a clamping position for grasping tissue between the jaw members 28 and 30 and against jaw member 30. Jaw member 30 serves as an active or oscillating blade and is configured to effect tissue. To this end, jaw member 30 includes an ultrasonic member (not shown) that is operably coupled to a transducer 24 (shown in phantom), and an operating surface 34 configured to effect tissue. In the illustrated embodiment, the operating surface 34 is configured to transect, dissect and/or coagulate tissue upon actuation of an activation button on remote 200 operably coupled to base 300 and generator 26 via data port 340.

Surgeon M can place instrument 10c into one of two modes, a low-power mode of operation and a high-power mode of operation, mattering on the button selected 225, 227, 231, or 229 (See FIGS. 2A-2C) on remote control 200. More particularly, activation button 225 or any other button 227, 231, or 229 is depressable to a first position for delivering low-power to the active jaw member 30 and a second position for delivering high-power to the active jaw member 30. In the first position, one or more audio or visual indicators may indicate to user that the activation button 225 is in the low-power mode. For example, and in one particular embodiment, an audio indicator may include a low-pitch, slow pulsating tone that indicates to surgeon M that the activation button 225 is in the first position or low power mode. Likewise, one or more audio or visual indicators (not shown) may indicate to user that the activation button is in the high-power mode, e.g., an audio indicator may include a high-pitch, fast pulsating tone that indicates to a user that the activation button 225 is in the second position or high power mode.

Transducer 24 (shown in phantom) is configured to convert electrical energy to mechanical energy that produces motion of a waveguide 22 disposed in operative communication with the active jaw member 308. When the transducer 24 and waveguide 22 are driven at a specific resonant frequency, they produce mechanical motion at the active jaw member 30. The electronics of generator 26 converts the electrical energy into a high voltage AC waveform which, in turn, drives the transducer 24. In one particular embodiment, the frequency of this AC waveform is the same as the resonant frequency of the waveguide 22 and transducer 24. As can be appreciated, the magnitude of the AC waveform includes a value that produces the proper amount of mechanical motion.

Arm 44 may also attach to a removable camera attachment 152 via coupling mechanism 38. The camera attachment includes camera 150 and one connector (not shown) of coupling mechanism 38. The camera attachment 152 may attach to any arm 40, 12, or 44 because of the coupling mechanism 38, which may be a universal adapter that allows each removable tip 400 or camera attachment 152 to attach to any and all necessary power and actuation requirements required of each removable tip 400 or camera attachment 152.

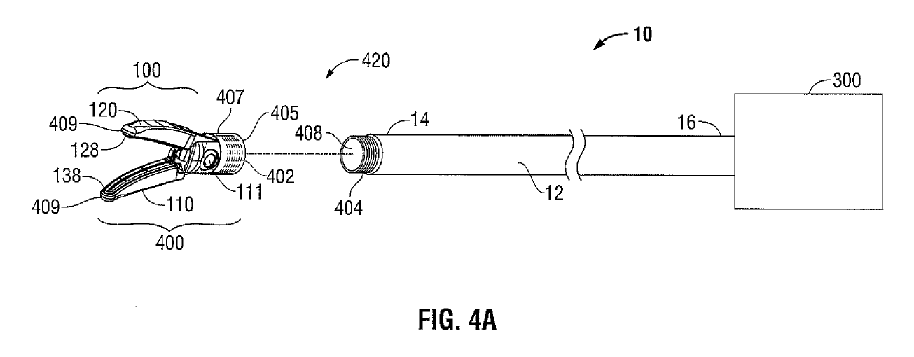

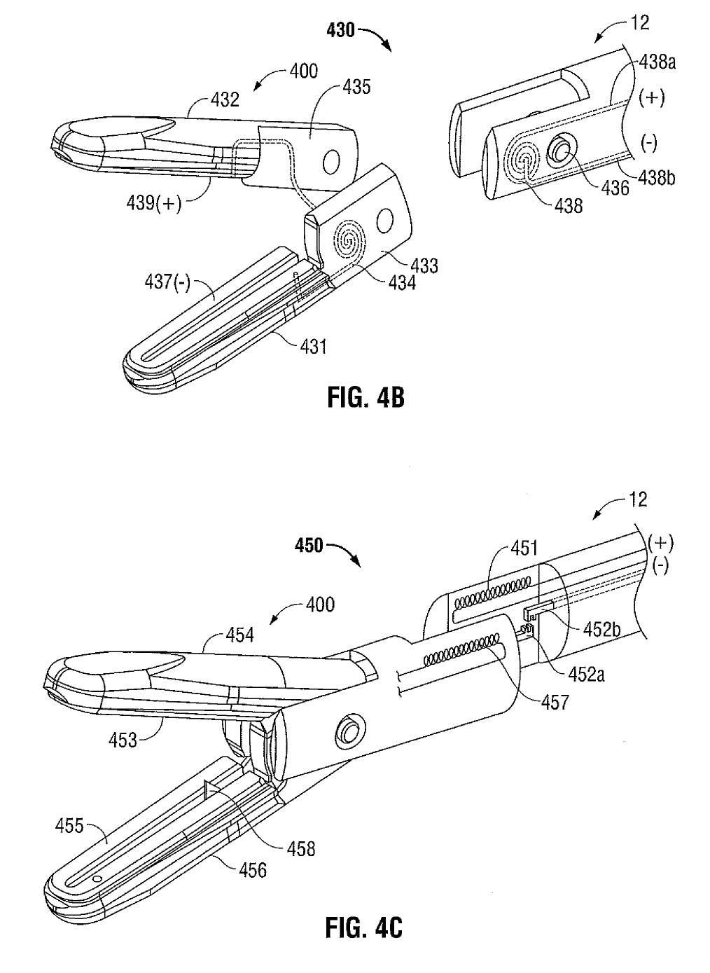

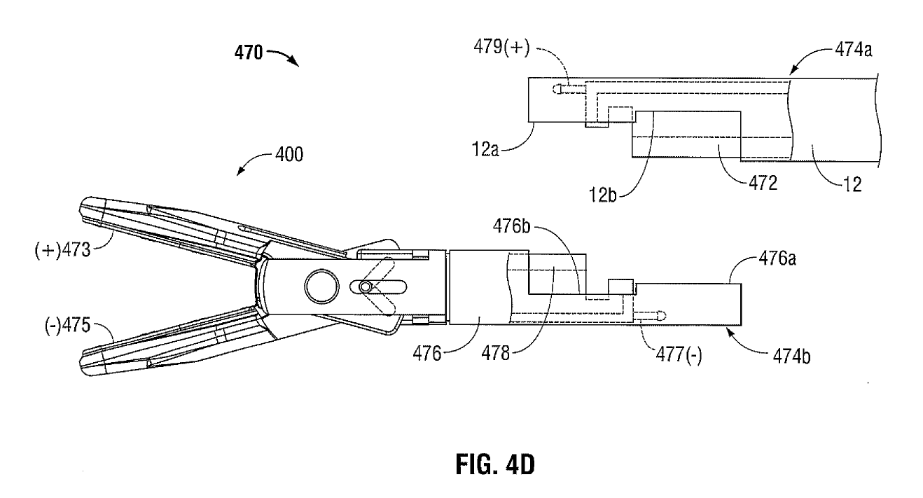

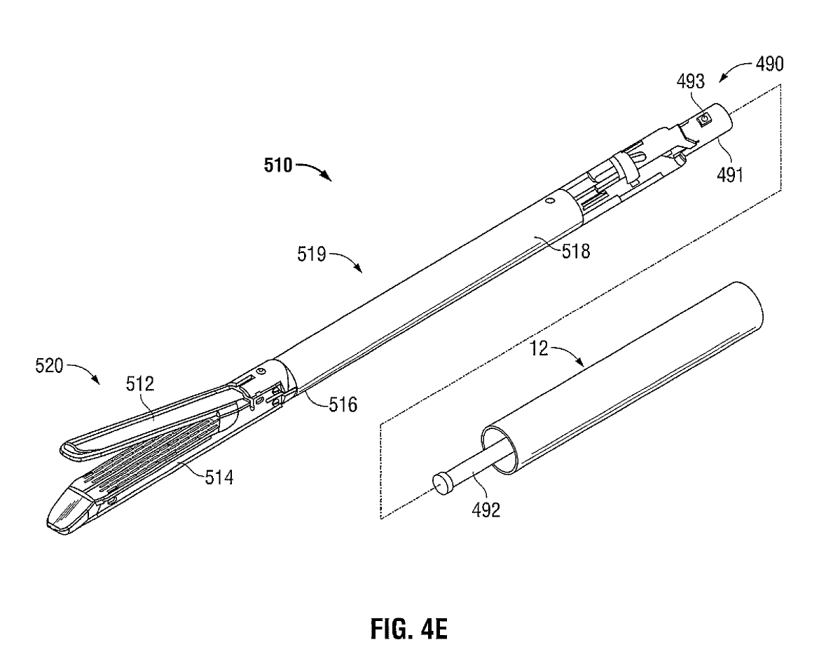

FIGS. 4A-4E show different coupling mechanisms 38 for coupling the removable tip 400 to arm 12. Any of the coupling mechanisms 38 may be used to attach arm 10 to base 300 when arm 12 is also removable. Referring to FIG. 4A, which shows a screw attachment coupling mechanism 420 for coupling removable tip 400 to arm 12. The removable tip 400 is configured to engage the distal end 14 of arm 12. Removable tip 400 includes a proximal end 407, a distal end 409, and a lumen 405 extending therethrough. Removable tip 400 may include threading 402 at proximal end 407 thereof for releasably engaging distal end 14 of arm 12 or may be configured to releasably engage distal end 14 of arm via any other suitable mechanism, e.g., snap-fit, friction-fit, etc. As can be appreciated, when removable tip 400 is engaged to distal end 14 of arm 12, lumen 405 of removable tip 400 is in communication with lumen 408 of arm 12 such that, power and actuation connections are coupled to allow the user M control of the removable tip 400. Additionally, mattering on the type of removable tip 400, fluid may be urged through lumen 408 of arm 12, into lumen 405 of removable tip 400 for application to an internal surgical site.

FIG. 4B shows an alternative coupling mechanism 430 using a spring-loaded pivot pin 436. The modular jaw members 432 and 431 are selectively removable from the arm 12 to facilitate replacement the jaw members 432, 431 following a surgical procedure. Pivot pin 436 may be spring-loaded to retain the flanges 435 and 433 within the bifurcated distal end of the arm 12 when the instrument 10 is in use. Following an electrosurgical procedure, the spring-loaded pivot pin 436 may be manipulated to release the used jaw members 432, 431 without requiring a cumbersome disassembly process. When the jaw members 432, 431 are connected to arm 12, a contactless electrical coupling is established. The proximal flange 433 of lower jaw member 431 is inductively-coupled to arm 12 through a pair of spiral coils 438, 434. The spiral coils 438, 434 form inductors, which store energy by generating a magnetic field when an electrical current is passed therethrough. The first coil 438 is disposed within arm 12, which forms part of a reusable base component of the instrument 10. The first coil 438 includes lead wires 438a, 438b extending through instrument 10. The second coil 434 is disposed on board the modular jaw member 431, which forms a replaceable component of removable tip 400. The second coil 434 is electrically coupled to the two electrodes 439 (+) and 437 (−) of opposite electrical potential.

Referring to FIG. 4C, shows an alternative coupling mechanism 450 using a reciprocal motion linkage 452a and 452b. To facilitate the mechanical motion of the jaw members 454, 456 and/or the knife blade 458, the removable tip 400 includes a first linkage 452a for receiving reciprocal motion from a corresponding second linkage 452b on arm 12. The first linkage 452a may be directly coupled to the knife 458 such that reciprocal motion of the first linkage 452a induces a corresponding reciprocal motion in the knife 458. Alternatively or additionally, the reciprocal motion of the first linkage may be converted to pivotal motion of the jaw members 456, 454 through the use of cam surfaces (not shown) or other conventional mechanisms. In addition, electrodes 453, 455 are powered by current flowing through second coil 457 from first coil 451 through inductive coupling. Another alternative coupling mechanism 38 is capacitive coupling disclosed in U.S. patent application Ser. No. 12/758,524, filed on Apr. 12, 2010, entitled “Surgical Instrument with Non-Contact Electrical Coupling”, the disclosure of which is herein incorporated by reference in its entirety.

Alternatively, the coupling mechanism 38 may be a separable coaxial joint 470 as shown in FIG. 4D. Both arm 12 and jaw drive shaft 472 exhibit an undercut profile at the distal end thereof. This profile permits each component 12 and 472 of the first mating component 474a to interlock with respective corresponding component of the second mating component 474b. Arm 12 exhibits an undercut profile as exemplified by a laterally prominent distal hook portion 12a and a laterally indented, recessed or undercut hook receiving portion 12b. A laterally prominent hook portion 476a of removable tip shaft 476 may be received in the hook receiving portion 12b, and a hook receiving portion 476b of the removable tip shaft 476 may receive the hook portion 12a of arm 12. When thus engaged, arm 12 and removable tip shaft 476 are axially aligned and resist longitudinal separation because of the interlocking hook portions 476a, 12a. Electrical connectivity may also be established by interlocking the first and second jaw drive shafts 472, 478. The first jaw drive shaft 472 includes an electrically conductive pin 479 protruding from a distal end thereof and an electrically conductive pin-receiving slot (not shown) on a lateral side thereof. The pin 479 and slot may be electrically coupled to opposite poles (+), (−) of the generator 26. The slot (−)(not shown) is configured to receive an electrically conductive pin 477(−) protruding from a proximal end of the distal jaw drive shaft 478. The electrically conductive pin 477(−) is in electrical communication with electrode 475(−). Similarly, the pin 479 may be electrically coupled to a slot (not shown) defined in the distal jaw drive shaft 478 to establish electrical continuity between electrode 473(+) and the generator 26.

Another alternative embodiment that may be used to as a coupling mechanism 38 to connect the removable tip 400 to arm 12 is an articulation link 490 as shown in FIG. 4E. In this embodiment, the removable tip 400 is shown as a removable stapler tip 510, that includes an end effector 520 attached to a mounting portion 516, which is pivotably attached to a body portion 518. Body portion 518 may provide a replaceable, disposable loading unit (DLU) or single use loading unit (SULU) (e.g., loading unit 519). In certain embodiments, the reusable portion may be configured for sterilization and re-use in a subsequent surgical procedure. End effector 520 includes anvil and the cartridge assemblies 512 and 514.

When the loading unit 519 is loaded into arm 12, the proximal portion 491 abuts a sensor tube (not shown) within firing arm 492, which displaces the sensor tube in a proximal direction. The movement of the sensor tube activates a switch in base 300 denoting that the loading unit 169 has been properly inserted. Coupling mechanism 38 may include one or more features that allow a control system 305 within base 300 to determine that removable tip 400 is properly inserted within arm 12, 40, or 44.

Various types of loading units 519 may include a protrusion 493 and/or extended insertion tips (not shown) for engaging the sensor tube. A non-articulating loading unit may include a protrusion 493 of a first type, while an articulating loading unit 519 may have a protrusion 493 of a second type that is of different dimensions that the first type protrusion 493. In other words, the protrusion 493 of one loading unit 519 is either longer or shorter than the protrusion 493 on another type of loading unit 519. As a result, when inserted, each type of the loading unit 519 engages the sensor tube by a predetermined distance. As a result, a variable loading unit sensor (not shown) then transmits the corresponding sensor signal corresponding to the displacement of the sensor tube to the control system 305, which then determines the type of the loading unit 519 based thereon. The control system 305 may then activate an articulation mechanism (not shown) when the loading unit 519 is of articulating type. Any coupling mechanism 38 disclosed herein may include a sensor (not shown), protrusion, and/or other similar feature within the removable tip 400 that indicates to the control system 305 the type of removable tip 400. The control system 305 then activates any features necessary to use that removable tip 400 and deactivates any features not necessary to use the removable tip 400.

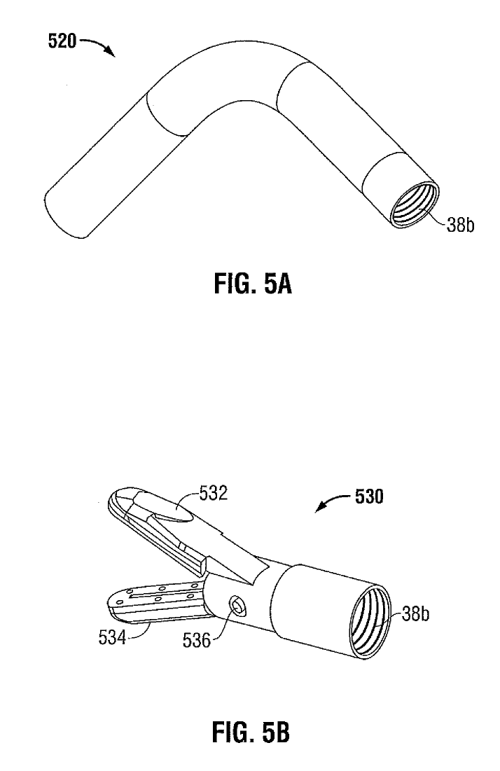

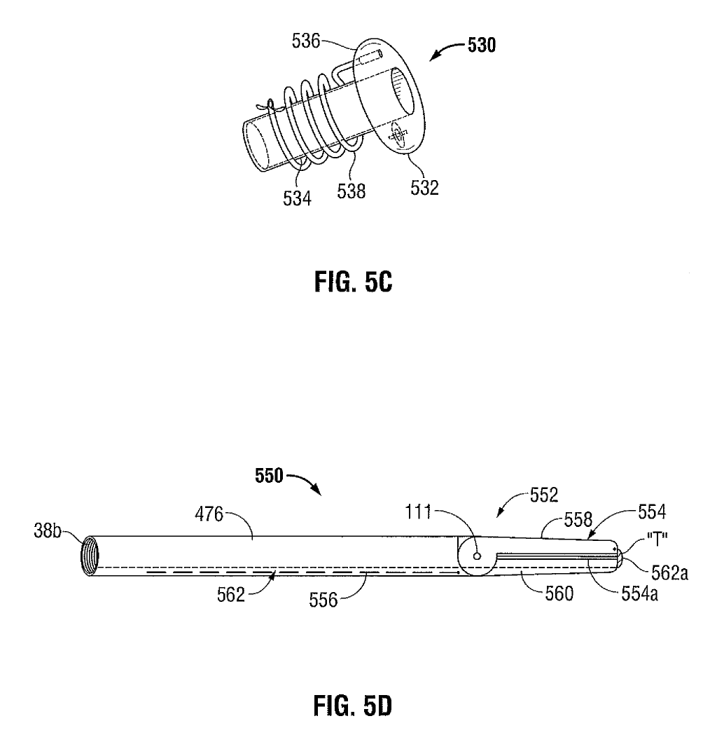

Removable tip 400 may be any type of diagnostic instrument. One example is an ultrasonic tip 410 shown FIG. 3B. The universal coupling mechanism 38 allows the ultrasonic tip 410 to connect to the transducer 24 and generator 26. Another type of tip is shown in FIG. 5A which shows a monopolar L hook tip 520 that connects to arm 12 with a mating component 38b. Alternatively, the removable tip 400 may be a grasper tip 530 as shown in FIG. 5B. The grasper tip includes a first jaw member 532 and second jaw member 534 that pivot together around pivot pin 536.

Alternatively, the removable tip 400 may be a suturing tip 530 as shown in FIG. 5C. Suturing tip 530 includes a housing 532 including an elongated tubular member 534 that extends distally from a distal end 536 of the housing 532. One or more helical needles 538 are positioned about the housing 532 and extend along a length of the elongated tubular member 534. One or more types of suture “S” (e.g., absorbable and/or nonabsorbable) operably couple to the suture tip 530. More particularly, suturing tip threads or positions suture “S” helically in tissue adjacent an opening, e.g., a wound, such that when a force (e.g., a “pulling” force) is applied to an accessible end of the suture “S” the opening closes.

In another embodiment, removable tip 400 may be a mechanical scissor tip 550 as shown in FIG. 5D. The mechanical scissor tip 550 includes an end effector assembly 552. End effector assembly 552 includes a knife or scissor blade 554 pivotably connected to removable tip shaft 476. Scissor blade 554 may be pivotably connected to the distal end of removable tip shaft 476 via pivot pin 111. Scissor blade 350 defines a cutting edge 554a or the like. A linkage 556 connects to a linkage (not shown) on arm 12 via coupling mechanism 38b to actuate scissor blade 554 relative to jaw members 558, 560 of end effector assembly 552 to sever tissue “T” and the like. End effector assembly 552 may further include a wire 562 extending out of one of jaw members 558, 560 and anchored to the other of jaw members 558, 560. In particular, wire 562 is disposed within central body portion 476 and includes a proximal end (not shown) which connects to a generator 24 via arm 12 and base 300, and a distal end 562a which extends out through fixed jaw member 560 and attaches to a distal end or tip of movable jaw member 558 to effect tissue.

In other embodiments, the removable tip may be a stapler tip 510 (See FIG. 4E), a Ligasure® tip, a monopolar needle, or any other type of end effector for a diagnostic or surgical procedure. The universal coupling mechanism 38 allows any tip 400 to be placed on any arm 12, 40, 44 and to allow all features of the removable tip 400 to be powered, actuated, or performed by base 300.

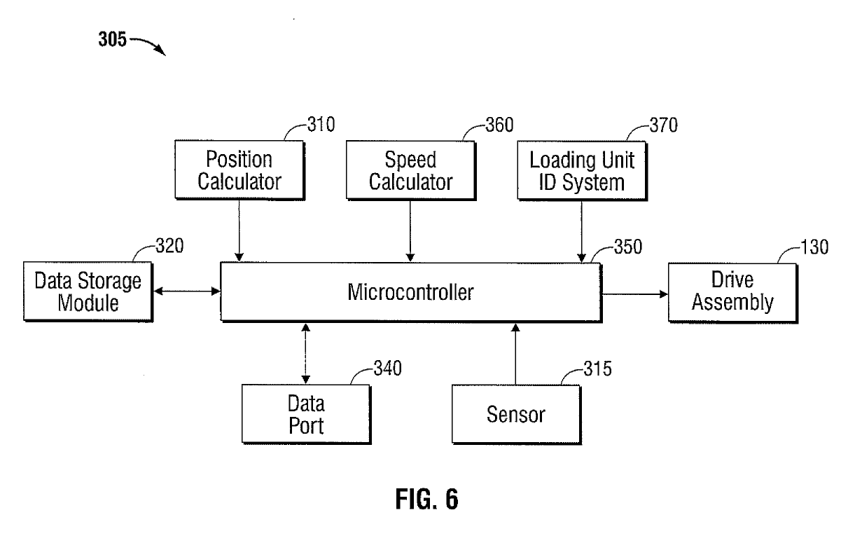

FIG. 6 illustrates a control system 305 for the RC surgical instrument 10 including the microcontroller 350 which is coupled to the position and speed calculators 310 and 360, the loading unit identification system 370, the drive assembly 130, and a data storage module 340. In addition, the microcontroller 350 may be directly coupled to a sensor 315, such as a motion sensor, torque meter, ohm meter, load cell, current sensor, etc. The microcontroller 350 includes internal memory which stores one or more software applications (e.g., firmware) for controlling the operation and functionality of the RC surgical instrument 10.

The loading unit identification system 370 identifies to the microcontroller 350 which removable tip 400 including end effector assembly 100 is attached to the distal end 14 of the RC instrument 10. In an embodiment, the control system 300 is capable of storing information relating to the force applied by the end effector assembly 100, such that when a specific end effector assembly 100 is identified the microcontroller 350 automatically selects the operating parameters for the RC surgical instrument 10. For example, torque parameters could be stored in data storage module 320 for a laparoscopic grasper. Additionally, microcontroller can activate or deactivate features within the base that are necessary or not necessary for the removable tip 400. For example, if the removable tip 400 is a Ligasure® tip, then at least the transducer 24 is deactivated and the drive assembly 130 is activated when the user M controls the Ligasure® tip.

The microcontroller 350 also analyzes the calculations from the position and speed calculators 310 and 360 and other sensors 315 to determine the actual position, direction of motion, and/or operating status of components of the RC surgical instrument 10. The analysis may include interpretation of the sensed feedback signal from the calculators 310 and 360 to control the movement of the drive assembly 130 and other components of the RC surgical instrument 10 in response to the sensed signal. Alternatively, the location of the RC surgical instrument 10 may be calculated using the method disclosed in U.S. Ser. No. 12/720,881, entitled “System and Method for Determining Proximity Relative to a Critical Structure” filed on Mar. 10, 2010, which is hereby incorporated by reference. The microcontroller 350 is configured to limit the travel of the end effector assembly 100 once the end effector assembly 100 has moved beyond a predetermined point as reported by the position calculator 310. Specifically, if the microcontroller determines that the position of the end effector assembly 100 is within a safety zone determined by the AR controller 200, the microcontroller is configured to stop the drive assembly 130.

In one embodiment, the RC surgical instrument 10 includes various sensors 315 configured to measure current (e.g., an ampmeter), resistance (e.g., an ohm meter), and force (e.g., torque meters and load cells) to determine loading conditions on the end effector assembly 100. During operation of the RC surgical instrument 10 it may be desirable to know the amount of force exerted on the tissue for a given end effector assembly 100. Detection of abnormal loads (e.g., outside a predetermined load range) may indicate a problem with the RC surgical instrument 10 and/or clamped tissue which is communicated to the user.

The data storage module 320 records the data from the sensors 315 coupled to the microcontroller 350. In addition, the data storage module 320 may record the identifying code of the end effector assembly 100, user of surgical tool, and other information relating to the status of components of the RC surgical instrument 10. The data storage module 320 is also configured to connect to an external device such as a personal computer, a PDA, a smartphone, or a storage device (e.g., a Secure Digital™ card, a CompactFlash® card, or a Memory Stick™) through a wireless or wired data port 340. This allows the data storage module 320 to transmit performance data to the external device for subsequent analysis and/or storage. The data port 340 also allows for “in the field” upgrades of the firmware of the microcontroller 350.

Embodiments of the present disclosure may include an augmented reality (AR) control system 610 as shown in FIGS. 7-8. The RC surgical instrument 10 is connected to an AR controller 600 via the data port 660 which may be either wired (e.g., FireWire®, USB, Serial RS232, Serial RS485, USART, Ethernet, etc.) or wireless (e.g., Bluetooth®, ANT3®, KNX®, Z-Wave®, X10®, Wireless USB®, Wi-Fi®, IrDA®, nanoNET®, TinyOS®, ZigBee®, 802.11 IEEE, and other radio, infrared, UHF, VHF communications and the like). Additionally, remote 200 (220, 240, 260) is connected to the AR controller 600 via data port 660 which may be either wired (e.g., FireWire®, USB, Serial RS232, Serial RS485, USART, Ethernet, etc.) or wireless (e.g., Bluetooth®, ANT3®, KNX®, Z-Wave®, X10®, Wireless USB®, Wi-Fi®, IrDA®, nanoNET®, TinyOS®, ZigBee®, 802.11 IEEE, and other radio, infrared, UHF, VHF communications and the like).

FIG. 7 illustrates a schematic diagram of an AR control system 610 in accordance with an embodiment of the present disclosure. With reference to FIG. 7, the augmented reality (AR) controller 600 is configured to store data transmitted to the controller 600 by a RC surgical instrument 10 and a remote 200 (220, 240, 260) as well as process and analyze the data. The RC surgical instrument 10 in this instance is a robotic instrument. The AR controller 600 may also connected to other devices, such as a video display 140, a video processor 120 and a computing device 180 (e.g., a personal computer, a PDA, a smartphone, a storage device, etc.). The video processor 120 may be used for processing output data generated by the AR controller 600 for output on the video display 140. Additionally, the video processor 120 may receive a real time video signal from a camera 150 inserted into the patient during the surgical procedure. Camera 150 may be a removable tip 152 attached to arm 44 or a stand alone camera 150. The computing device 180 may be used for additional processing of the pre-operative imaged data. In one embodiment, the results of pre-operative imaging such as an ultrasound, MRI, x-ray, or other diagnosing image may be stored internally for later retrieval by the computing device 180.

The AR controller 600 includes a data port 660 (FIG. 8) coupled to the microcontroller 650 which allows the AR controller 600 to be connected to the computing device 180. The data port 660 may provide for wired and/or wireless communication with the computing device 180 providing for an interface between the computing device 180 and the AR controller 600 for retrieval of stored pre-operative imaging data, configuration of operating parameters of the AR controller 600 and upgrade of firmware and/or other software of the AR controller 600.

Components of the AR controller 600 are shown in FIG. 8. The AR controller 600 includes a microcontroller 650, a data storage module 655 a user feedback module 665, an OSD module 640, a HUD module 630, and a data port 660.

The data storage module 655 may include one or more internal and/or external storage devices, such as magnetic hard drives, or flash memory (e.g., Secure Digital® card, Compact Flash® card, or MemoryStick®). The data storage module 655 is used by the AR controller 600 to store data from the RC surgical instrument 10 and remote 200 (220, 240, 260) for later analysis of the data by the computing device 180. The data may include information supplied by a sensor 315 (FIG. 6), such as a motion sensor, torque sensor, and other sensors disposed within the RC surgical instrument 10.

The microcontroller 650 may supplant, complement, or supplement the control circuitry 305 of the RC surgical instrument 10 shown in FIG. 6. The microcontroller 650 includes internal memory which stores one or more software applications (e.g., firmware) for controlling the operation and functionality of the RC surgical instrument 10. The microcontroller 650 processes input data from the computing device 180 and adjusts the operation of the RC surgical instrument 10 in response to the inputs. The RC surgical instrument 10 is configured to connect to the AR controller 600 wirelessly or through a wired connection via a data port 340. The microcontroller 650 is coupled to the user feedback module 665 which is configured to inform the user of operational parameters of the RC surgical instrument 10. The user feedback module 665 may be connected to a user interface. The user feedback module 665 may be coupled to the haptic mechanism 232 within the remote 200 (220, 240, 260) to provide for haptic or vibratory feedback. The haptic feedback may be used in conjunction with the auditory and visual feedback or in lieu of the same to avoid confusion with the operating room equipment which relies on audio and visual feedback. The haptic mechanism 232 may be an asynchronous motor that vibrates in a pulsating manner. In one embodiment, the vibrations are at a frequency of about 30 Hz or above. The haptic feedback can be increased or decreased in intensity. For example, the intensity of the feedback may be used to indicate that the forces on the instrument are becoming excessive. In alternative embodiments, the user feedback module 265 may also include visual and/or audible outputs.

The microcontroller 650 outputs data on video display 140 and/or the heads-up display (HUD) 635. The video display 140 may be any type of display such as an LCD screen, a plasma screen, electroluminescent screen and the like. In one embodiment, the video display 140 may include a touch screen and may incorporate resistive, surface wave, capacitive, infrared, strain gauge, optical, dispersive signal or acoustic pulse recognition touch screen technologies. The touch screen may be used to allow the user to provide input data while viewing AR video. For example, a user may add a label identifying the surgeon for each tool on the screen. The HUD display 635 may be projected onto any surface visible to the user during surgical procedures, such as lenses of a pair of glasses and/or goggles, a face shield, and the like. This allows the user to visualize vital AR information from the AR controller 600 without loosing focus on the procedure.

The AR controller 600 includes an on-screen display (OSD) module 640 and a HUD module 630. The modules 640, 630 process the output of the microcontroller 650 for display on the respective displays 140 and 635. More specifically, the OSD module 640 overlays text and/or graphical information from the AR controller 600 over video images received from the surgical site via camera 150 (FIG. 1) disposed therein. Specifically, the overlaid text and/or graphical information from the AR controller 600 includes computed data from pre-operative images, such as x-rays, ultrasounds, MRIs, and/or other diagnosing images. The computing devices 180 stores the one or more pre-operative images. In an alternative embodiment, the data storage module 655 can store the pre-operative image. The AR controller 600 processes the one or more pre-operative images to determine margins and location of an anatomical body in a patient, such as an organ or a tumor. Alternatively, the computing device 180 can process and analyze the pre-operative image. Additionally, the AR controller can create safety boundaries around delicate structures, such as an artery or organ. Further, the AR controller 600 can decipher the one or more pre-operative images to define structures, organs, anatomical geometries, vessels, tissue planes, orientation, and other similar information. The AR controller 600 overlays the information processed from the one or more pre-operative images onto a real time video signal from the camera 150 within the patient. The augmented video signal including the overlaid information is transmitted to the video display 140 allowing the user to visualize more information about the surgical site including area outside the vision of the camera 150. Additionally, as the camera moves around the surgical site, the labels and/or data overlaid is moved to the appropriate location on the real time video signal.

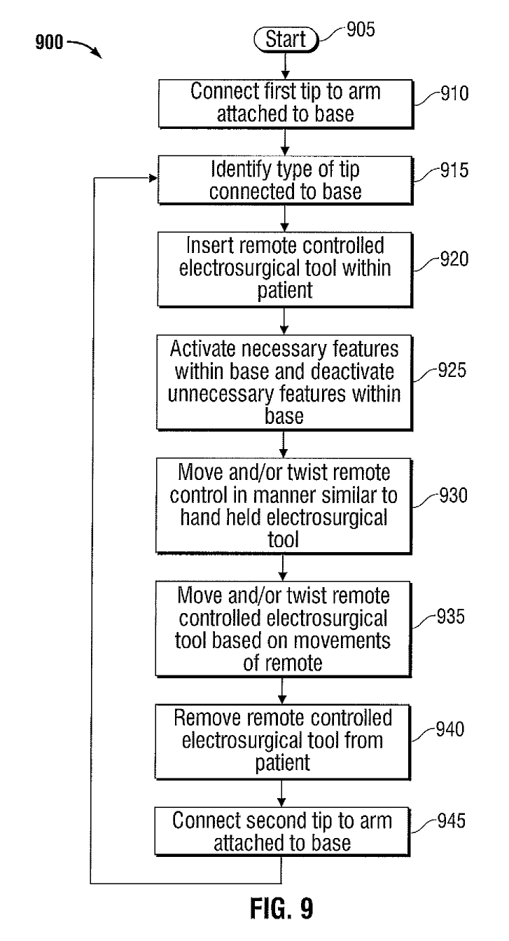

FIG. 9 is a flow diagram of a process 900 for setting up and controlling an electrosurgical instrument with removable tips 400 according to an embodiment of the invention. After the process 900 starts at step 905, a first removable tip 400 is attached to arm 12 at step 910. Arm 12 may be attached permanently to base 300 or removably coupled to base 300. Additionally, when multiple arms 12, 40, 44 are attached to base 300, then multiple removable tips 400 may be attached in step 905. Next at step 915, control system 305 determines the type of removable tip 400 attached to base 300 via arm 12. The control system 305 may read a sensor (not shown) or determine a physical change from a protrusion 493 or other feature within tip 400. A RC electrosurgical instrument 10 is inserted into a body cavity or incision at step 920. Before or after the RC electrosurgical instrument 10 is inserted within the patient P, the control system 305 determines which features to activate and which features to deactivate based on the type of removable tip. For example, if removable tip 400 is an ultrasonic tip, then the transducer 24 is activated and the drive assembly 130 is deactivated. A user M moves, twists, and/or selects buttons on the remote control 200 at step 930. The surgeon M may move the remote 200 in a manner similar to actions done with a handheld electrosurgical instrument. The RC surgical instrument 10 moves, twist, and/or performs other action based on the movements performed by the remote 200 at step 935. The movements of the remote 200 are sent wirelessly or the remote is directly connected to the RC surgical instrument 10 via box 300 (See FIG. 3A previously called “base”). Next, at step 940, the RC electrosurgical instrument 10 is removed from the patient P. Then, the first removable tip is removed and the second removable tip is coupled to arm 12 at step 915. The procedure returns to step 915 to identify type of tip the second removable tip is. The procedure 900 completes when the surgical procedure is complete.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A remote controlled electrosurgical instrument assembly, comprising:
a base configured with a transducer and a drive assembly;
an arm connected to the base;
a tip removably coupled to the arm; and
a remote control configured to send a plurality of instructions to the base to control motions of the tip and for the base to supply an electrical signal to the tip.

2. The remote controlled electrosurgical instrument assembly according to claim 1, wherein the electrical signal supplied from the base is at least one of an RF signal and an ultrasonic signal.

3. The remote controlled electrosurgical instrument assembly according to claim 1, wherein the tip is a monopolar loop, a monopolar “L” hook tip, a coagulation tip, a mechanical scissor, a grasper, a camera, a suturing tip, a stapling tip, or other type of diagnostic instrument.

4. The remote controlled electrosurgical instrument assembly according to claim 1, wherein the tip includes an identifying sensor to indicate the type of tip.

5. The remote controlled electrosurgical instrument assembly according to claim 4, wherein the base includes a control system and the control system is configured to read the identifying sensor within the tip and determine the type of tip and disengage any functions within the base that are not used by the respective tip.

6. The remote controlled electrosurgical instrument assembly according to claim 1, wherein the tip is coupled to the arm by a screw attachment mechanism, a spring loaded pivot pin, a reciprocal motion linkage, a capacitive coupling mechanism, a separable coaxial joint, or an articulation link.

7. The remote controlled electrosurgical instrument assembly according to claim 1, wherein the tip is coupled to arm and the base using a universal interface that allows any tip attached to the arm to share power, jaw drivers, and the ultrasonic transducers.

8. The remote controlled electrosurgical instrument assembly according to claim 1, wherein the base includes a generator for supplying power to the tip.

9. The remote controlled electrosurgical instrument assembly according to claim 1, wherein the base is attached to a generator for supplying power to the tip.

10. The remote controlled electrosurgical instrument assembly according to claim 1, wherein the tip is reuseable or disposable.

11. The remote controlled electrosurgical instrument assembly according to claim 1, wherein the tip includes one or more sensors that function in a closed loop feedback circuit to control motion of tip by the base and the remote control.

12. A remote controlled electrosurgical instrument assembly, comprising:
a base configured with a transducer and a drive assembly;
a first and second arm each connected to the base;
a first tip removably coupled to the first arm, wherein the first tip includes an ultrasonic end effector;
a second tip removably coupled to the second arm, wherein the second tip includes a RF end effector;
a remote control configured to send a plurality of instructions to the base to control motions of the first tip and second tip, wherein the base supplies an ultrasonic signal to the first tip when instructed by the remote control and supplies a RF signal to the second tip when instructed by the remote control.

13. The remote controlled electrosurgical instrument assembly according to claim 12, wherein the first and second tip each include an identifying sensor.

14. The remote controlled electrosurgical instrument assembly according to claim 13, wherein the base includes a control system and the control system reads the sensor within the first tip and deactivates the drive assembly when the remote control is controlling the first tip.

15. The remote controlled electrosurgical instrument assembly according to claim 13, wherein the base includes a control system and the control system reads the sensor within the second tip and deactivates the transducer when the remote control is controlling the second tip.

16. The remote controlled electrosurgical instrument assembly according to claim 12, further comprising a third arm and the third arm includes a camera tip.

17. The remote controlled electrosurgical instrument assembly according to claim 12, further comprising a camera tip attached to the first arm when the first tip is removed.

18. A method for performing an electrosurgical procedure, the method comprising:
inserting a remote controlled electrosurgical instrument within a patient, wherein the remote controlled electrosurgical instrument is configured with a base, an arm, and a removable tip coupled to the arm;
moving a remote in a manner substantially similar to movement of a handheld electrosurgical instrument, wherein the remote is configured with at least one momentum sensor;
sending information from the momentum sensor to the base to move the remote controlled electrosurgical instrument within the patient based on movements of the remote;
removing the tip from the arm;
coupling a second tip to the arm;
reading a sensor within the second tip to identify the type of tip; and
deactivating functions within the base not used by the second tip.

19. The method according to claim 18, further comprising:
storing a pre-operative image of an anatomical section of the patient;
analyzing the pre-operative image to determine a safety zone around an anatomical body within the patient, wherein the anatomical body is located within the anatomical section;
receiving a video signal from a camera located within the patient during a surgical procedure;
augmenting the safety zone onto the video signal;
displaying the video signal with the safety zone;
measuring a location of the remote controlled electrosurgical instrument within the patient;
determining if the remote controlled electrosurgical instrument is within the safety zone; and
in response to determining the remote controlled electrosurgical instrument is within the safety zone, generating a notification to the user or stopping a drive motor within the remote controlled electrosurgical instrument, wherein the feedback can be increased based on location of the instrument within the safety zone.

20. The method according to claim 18, wherein the functions within the base include at least one of a transducer, jaw drive, and a power supply.
